(19) United States
(12) Patent Application Publication
(10) Pub. No.: US 2025/0295939 A1
(43) Pub. Date: Sep. 25, 2025

(54) HEATING DEVICE FOR IMPOSING INTERMITTENT THERMAL EFFECT ON NEURAL TISSUE OF ANIMAL OR HUMAN BODY IN VIVO

(71) Applicant: National Taiwan University, TAIPEI CITY (TW)

(72) Inventors: Chih-Yu CHAO, Taipei City (TW); Yu-Yi KUO, Taipei City (TW); Wei-Ting CHEN, Tainan City (TW); Guan-Bo LIN, Taipei City (TW); You-Ming CHEN, New Taipei City (TW); Hsu-Hsiang LIU, Taipei City (TW)

(73) Assignee: National Taiwan University, TAIPEI CITY (TW)

(21) Appl. No.: 18/864,192

(22) PCT Filed: Dec. 26, 2022
(86) PCT No.: PCT/CN2022/141921

§ 371 Date: Nov. 8, 2024

(51) Int. Cl. A61N 7/02; A61N 7/00; G16H 20/40

(52) U.S. Cl. CPC A61N 7/02; G16H 20/40; A61N 2007/0026

(57) ABSTRACT

A heating device for imposing intermittent thermal effect on neural tissue of an animal or a human body in vivo, including: a control unit having an executable thermal effect logic; a heater, wherein the thermal effect logic includes: sequentially, at least one first heating session, a first cooling session following the first heating session, and a second heating session following the first cooling session; in the first and second heating sessions a heated area stays between 39° C. and 42° C.; in the first cooling session the heated area is cooled down to below 39° C. and stays between 37° C. and 39° C.

### BACKGROUND OF THE INVENTION

### 1. Field of the Invention

The present disclosure relates to devices for imposing thermal effect on an animal or a human body in vivo, and more particularly to a heating device for imposing intermittent thermal effect on neural tissue of an animal or a human body in vivo.

### 2. Description of Related Art

Taiwan's patent I728589 discloses Method and Apparatus for Applying Heat to Living Tissue, characterized by heating and cooling tissue in vivo cyclically and keeping the heating temperature above 39° C. during a thermal effect time period that starts as soon as the heating process begins and during all heating and cooling stages to impose thermal effect on cells and excite heat shock proteins in cells to thereby bring about different effects on different cells, for example, recovery effect on normal cells or apoptosis effect on cancer cells.

The aforesaid prior art can achieve the anticipated effects. However, the temperature of the thermal effect imposed on an animal in vivo must always be kept above 39° C., and in consequence injuries are possible to occur because of the prolonged thermal effect imposed on neural tissue or cells. Therefore, it is necessary to provide an effective solution to the aforesaid issue without causing any injuries.

In addition, if it is feasible to lower the temperature during a thermal effect time period and still maintain the aforesaid advantages, it can be reasonably inferred that the thermal effect can last longer but does not or is unlikely to cause injuries to neural tissue.

### BRIEF SUMMARY OF THE INVENTION

It is an objective of the disclosure to provide a heating device for imposing intermittent thermal effect on neural tissue of an animal or a human body in vivo, especially imposing intermittent thermal effect on an animal in vivo at a temperature lower than a conventional temperature to not only allow the thermal effect to last longer but also cause no injuries to neural tissue.

To achieve the above and other objectives, the disclosure provides a heating device for imposing intermittent thermal effect on neural tissue of an animal or a human body in vivo, comprising: a control unit having a thermal effect logic to be executed by the control unit; and a heater electrically connected to the control unit, the control unit executing the thermal effect logic to controllably cause the heater to heat up a portion of neural tissue of an animal or a human body in vivo, wherein an area to be heated is defined as a heated area, wherein the thermal effect logic comprises: sequentially, at least one first heating session is followed by a first cooling session, and the first cooling session is followed by a second heating session; in the first or second heating session the control unit controllably causes the heater to perform a heating process; in the first cooling session the control unit controllably causes the heater to not perform a heating process or to perform a heating process with low power, wherein the heating process performed with low power is characterized in that it uses heating power lower than the heating power used in the first heating session; in the first heating session the heated area is heated up until the temperature of the heated area increases to above 39° C. and stays between 39° C. and 42° C., with the first heating session lasting for 3 to 60 minutes; in the first cooling session the heated area is cooled down to below 39° C. and stays between 37° C. and 39° C., with the first cooling session lasting for 5 seconds to 3 minutes; and in the second heating session the heated area is heated up to above 39° C. and stays between 39° C. and 42° C., with the second heating session lasting for 3 to 60 minutes, wherein the first heating session and the second heating session each last longer than the first cooling session.

Therefore, the disclosure is capable of imposing intermittent thermal effect on an animal in vivo at a temperature lower than a conventional temperature to not only allow the thermal effect to last longer but also cause no injuries to neural tissue.

### BRIEF DESCRIPTION OF THE DRAWINGS

FIG. 1 is a block diagram of the first preferred embodiment of the disclosure.

FIG. 2 is an installation schematic view of the first preferred embodiment of the disclosure.

FIG. 3 is a thermal effect temperature setting diagram of the first preferred embodiment of the disclosure.

FIG. 4 is a thermal effect result diagram of the first preferred embodiment of the disclosure.

FIG. 5 is a thermal effect schedule of the first preferred embodiment of the disclosure.

FIG. 6 is a Y-maze result diagram of the first preferred embodiment of the disclosure.

FIG. 7A is a statistical result diagram of the first preferred embodiment of the disclosure, showing Western blots of beta-amyloid peptide and β-actin and their ratio statistical result.

FIG. 7B is another statistical result diagram of the first preferred embodiment of the disclosure, showing Western blots of β-site amyloid precursor protein cleaving enzyme 1 and β-actin and their ratio statistical result.

FIG. 8A is yet another statistical result diagram of the first preferred embodiment of the disclosure, showing Western blots of ionized calcium-binding adapter molecule 1 and β-actin and their ratio statistical result.

FIG. 8B is still yet another statistical result diagram of the first preferred embodiment of the disclosure, showing Western blots of glial fibrillary acidic protein and β-actin and their ratio statistical result.

FIG. 9A is a further statistical result diagram of the first preferred embodiment of the disclosure, showing Western blots of insulin degrading enzyme and β-actin and their ratio statistical result.

FIG. 9B is a further statistical result diagram of the first preferred embodiment of the disclosure, showing Western blots of superoxide dismutase and β-actin and their ratio statistical result.

FIG. 10 is a block diagram of the second preferred embodiment of the disclosure.

FIG. 11 is a thermal effect temperature setting diagram of the second preferred embodiment of the disclosure.

FIG. 12 is a thermal effect temperature setting diagram of the third preferred embodiment of the disclosure.

FIG. 13 is another thermal effect temperature setting diagram of the third preferred embodiment of the disclosure.

### DETAILED DESCRIPTION OF THE INVENTION

The technical features of the disclosure are herein illustrated with preferred embodiments, depicted with drawings, and described below.

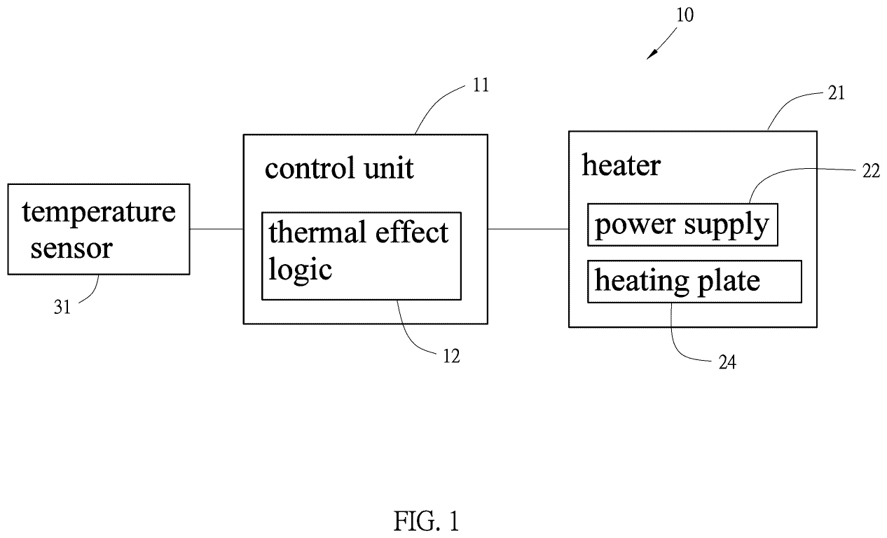

As shown in FIG. 1 through FIG. 9B, the first preferred embodiment of the disclosure provides a heating device 10 for imposing intermittent thermal effect on neural tissue of an animal or a human body in vivo, essentially comprising a control unit 11, a heater 21 and a temperature sensor 31.

The control unit 11 has a thermal effect logic 12 which can be executed by the control unit 11.

The heater 21 is electrically connected to the control unit 11. The control unit 11 executes the thermal effect logic 12 to controllably cause the heater 21 to heat a portion of neural tissue of an animal in vivo, and the area to be heated is defined as a heated area 92. The heater 21 is a well-known, existing, commercially-available heating device, for example, a focused ultrasound (FUS) device, steam and water bath, infrared device, radio frequency electromagnetic wave, microwave or heating pad device. The first embodiment is exemplified by a patch-style heating device. Therefore, in the first embodiment, the heater 21 has a power supply 22 and a heating plate 24. The power supply 22 is electrically connected to and thus controlled by the control unit 11 to provide electric power to the heating plate 24 for generating heat. In addition, during the heating process, the power supply 22 is controlled to provide a direct current (DC) or an alternating current (AC) power to the heating plate 24. In the first embodiment, the power supply 22 provides the direct current (DC) power, for exemplary purposes. In addition, the power supply 22 is used to adjust power to perform the heating process continuously, or a combination of a conventional function generator (not shown) and a power amplifier (not shown) provides pulse current to the heating plate 24 to generate heat pulse energy, wherein overall heating energy can be regulated by adjusting pulse frequency and duration.

In addition, if the heater 21 is a focused ultrasound device, it will generate ultrasound to provide heat pulse energy, enable the heat pulse energy to penetrate the skin of an animal or a human body in vivo through focusing technology, and thus enable the heat pulse energy to be focused on the interior neural tissue of the animal or human body, allowing the heat pulse energy to directly enter the animal or human body and thereby directly act on the heated area 92. The frequency of the heat pulse energy ranges from 5 Hz to 500 Hz, preferably from 25 Hz to 50 Hz. When it comes to one single frequency, it can be 40 Hz for use in stimulating neural cells with gamma wave frequency by a conventional means.

The temperature sensor 31 is electrically connected to the control unit 11 and adopted to sense the temperature of the heated area 92. The temperature sensor 31 is an existing, non-contact temperature-sensing device, for example, ultrasound temperature-sensing device, infrared temperature-sensing device, and nuclear magnetic resonance (NMR) temperature-sensing device. Alternatively, the temperature sensor 31 is a conventional contact-enabling temperature sensing device, for example, thermocouple. This embodiment is exemplified by a thermocouple for measuring the temperature of the heated area 92.

The thermal effect logic 12 comprises four parts as follows:

First, sequentially, at least one first heating session H1 is followed by a first cooling session C1, and the first cooling session C1 is followed by a second heating session H2. In the first or second heating session (H1 or H2), the control unit 11 controllably causes the heater 21 to perform a heating process. In the first cooling session C1, the control unit 11 controllably causes the heater 21 to not perform any heating process or to perform a heating process with low power; the heating process performed with low power is characterized in that it uses heating power lower than the heating power used in the first heating session H1.

Second, in the first heating session H1, the heated area 92 is heated up until the temperature of the heated area 92 increases to above 39° C. and stays between 39° C. and 42° C., with the first heating session H1 lasting for 3 to 60 minutes.

Third, in the first cooling session C1, the heated area 92 is cooled down to below 39° C. and stays between 37° C. and 39° C., with the first cooling session C1 lasting for 5 seconds to 3 minutes.

Fourth, in the second heating session H2, the heated area 92 is heated up to above 39° C. and stays between 39° C. and 42° C., with the second heating session H2 lasting for 3 to 60 minutes.

Therefore, the first heating session H1 and the second heating session H2 each last longer than the first cooling session C1.

In both the first heating session H1 and the second heating session H2, the heated area 92 is heated up to stay between 39° C. and 42° C., because neural tissue must be heated up to trigger the expression and effect of heat shock proteins to thereby stimulate the neural tissue and trigger recovery effect; however, the duration of the first heating session H1 and the second heating session H2 must not be longer than 60 minutes. Furthermore, the first heating session H1 and the second heating session H2 must not last for a time period that is overly short, for example, shorter than 3 minutes, in order to prevent the deterioration of the recovery effect. The first cooling session C1 allows the neural tissue to take a break at a lower temperature, otherwise the neural tissue might be heated up continuously for a long time period and exposed to excessive thermal stress and irritation to thereby get injured; however, the duration of the break must not be longer than 3 minutes in order to maintain the transmission of biochemical signals of heat shock proteins and other related proteins. Furthermore, the first cooling session C1 must not last for a time period that is overly short, for example, shorter than 5 seconds, in order to prevent the inadequacy of the break.

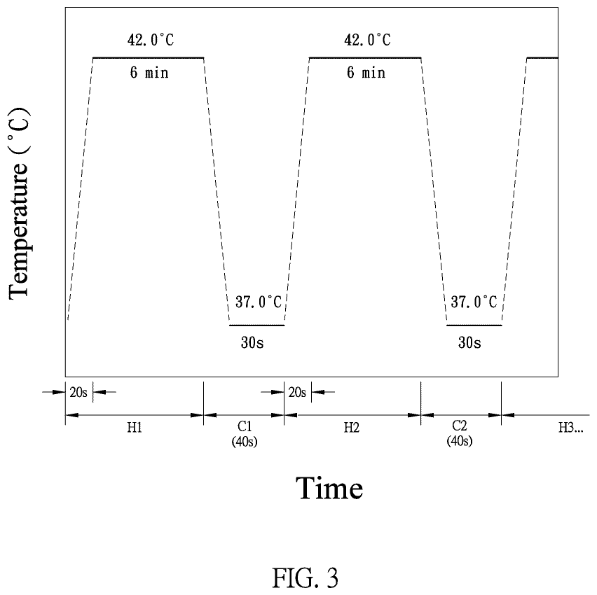

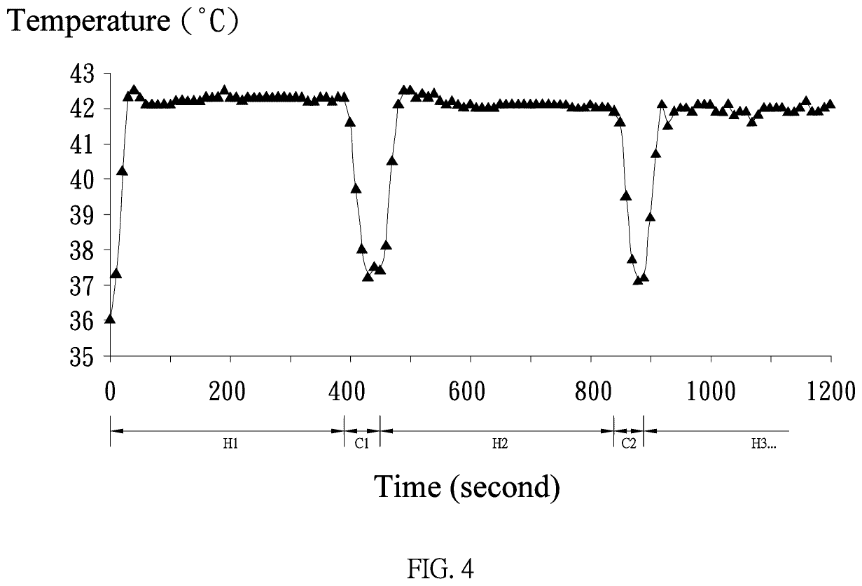

As shown in FIG. 3 and FIG. 4, in the first embodiment, in both the first heating session H1 and the second heating session H2, the heated area 92 is heated up to 42° C. and then stays at 42° C. As shown in FIG. 4, tiny temperature fluctuations occur in practice. Owing to the blood circulation of an animal in vivo or any other physiological factors, the heated area 92 will cool down rapidly to the normal body temperature unless it is continuously heated up. Therefore, this stage, in which the temperature of the heated area 92 is maintained at 42° C., must undergo heating continuously to allow the heated area 92 to stay at 42° C.; thus, this stage is a stage of dynamic equilibrium of heating and cooling. Therefore, in practice, the aforesaid temperature varies slightly with time and thus rarely, desirably stays at 42° C. constantly.

Moreover, in the first embodiment, the first heating session H1 and the second heating session H2 last for 6 minutes 20 seconds for exemplary purposes, during which the heated area 92 takes 20 seconds to be heated up to 42° C. and then stays at 42° C. for 6 minutes. For the sake of clarity, FIG. 3, FIG. 4 and FIG. 11 through FIG. 13 are not drawn to scale but are numerically expressed. In the first embodiment, the first cooling session C1 lasts for 40 seconds for exemplary purposes, during which the heated area 92 takes 10 seconds to be cooled down to 37° C. and then stays at 37° C. for 30 seconds.

In the first embodiment, during the first heating session H1 and the second heating session H2, the heated area 92 is preferably heated up to a target temperature in 2 minutes, and the target temperature in the first embodiment is 42° C. During the process of heating up the heated area 92 to the target temperature, it is feasible to heat up the heated area 92 to a temperature higher than the target temperature by no greater than 0.5° C. and then reduce the heating power of the heater 21 to cause the temperature of the heated area 92 to decrease and stay at the target temperature.

In addition, it is feasible that the second heating session H2 is followed by an additional cooling session and an additional heating session to meet user needs. For example, the second heating session H2 is followed by a second cooling session C2 and a third heating session H3 or more cooling sessions and heating sessions, as shown in FIG. 3 and FIG. 4.

The structural and technical features of the first embodiment are described above. The operation states of the first embodiment are described below.

Since conventional biological experiments are mostly conducted with in vivo mouse model, a live mouse is used in the first embodiment for exemplary purposes.

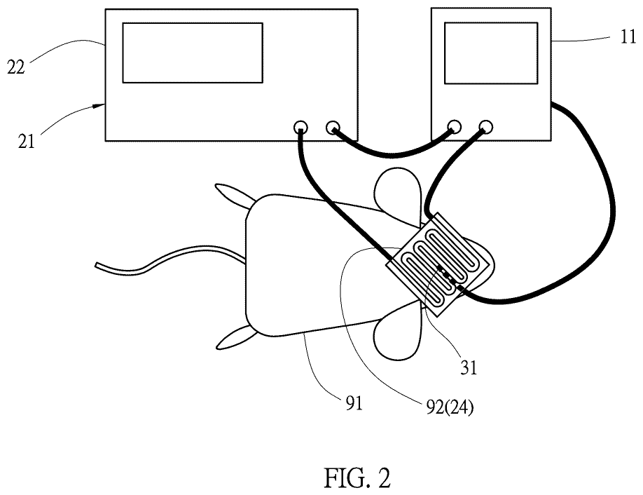

As shown in FIG. 2, during the operation, the heating plate 24 of the heater 21 is attached to the top of the head of a mouse 91, and the temperature sensor 31 is located at the top of the head of the mouse 91 but under the heating plate 24 to measure temperature. The top of the head of the mouse 91, which is located under the heating plate 24, is regarded as the heated area 92. Then, the control unit 11 executes the thermal effect logic 12 to achieve the following: in the first heating session H1 the heated area 92 is heated up to 42° C. in around 20 seconds and then stays at 42° C. for 6 minutes before the first cooling session C1 begins; in the first cooling session C1 the heated area 92 is cooled down to 37° C. in around 10 seconds and then stays at 37° C. for 30 seconds before the second heating session H2 begins. As shown in FIG. 3 and FIG. 4, the second heating session H2 is followed by a second cooling session C2 and a third heating session H3, and so forth. The first embodiment provides a total of 10 heating sessions and a total of 9 intervening cooling sessions, and such a heating process is defined as “ten thermal cycles”.

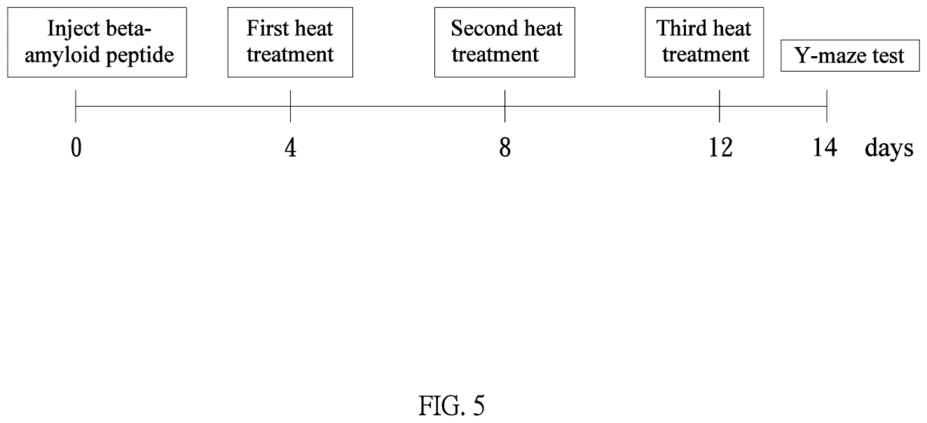

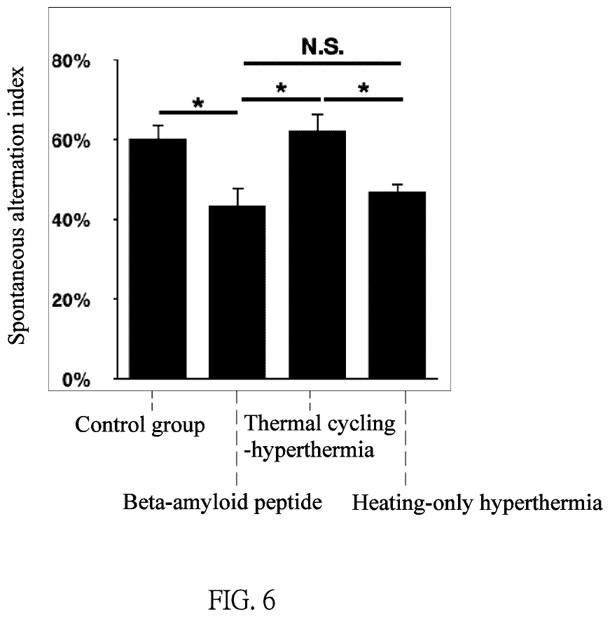

As shown in FIG. 5, from the very beginning, a beta-amyloid peptide (Aβ) solution is injected into the hippocampus of a mouse brain, and the solution is 10 microliters in volume and 1 mg/cc in concentration. The aforesaid ten thermal cycles are defined as a single thermal cycling-hyperthermia (TC-HT) treatment. The heat treatment using TC-HT is performed on the mouse three times within the 14 days of test schedule. After that, a Y-maze test is performed on the mouse on the 14th day. The aforesaid “performing TC-HT treatment three times within 14 days” is defined as entire thermal cycling-hyperthermia (TC-HT) treatment and thus can be used as a potential alternative therapeutic method for treating dementia. The first embodiment involves comparing thermal cycling-hyperthermia (TC-HT) with conventional continuous hyperthermia (HT) in terms of the therapeutic efficacy in the treatment of the mouse with dementia. The effect resulting from the test conducted on the mouse is shown in FIG. 6. FIG. 6 demonstrates the results of spontaneous alternation indexes when the Y-maze test is conducted in four test groups as follows: control group (Ctrl); injecting beta-amyloid peptide (Aβ) into mouse hippocampus to simulate mouse dementia (as denoted by “beta-amyloid peptide” in FIG. 6); performing thermal cycling-hyperthermia (TC-HT) on the mouse with dementia for three times (as denoted by “thermal cycling-hyperthermia” in FIG. 6); and performing continuous hyperthermia (HT) on the dementia mouse three times (as denoted by “continuous hyperthermia” in FIG. 6). Furthermore, additional result of the test is that the mouse made the same number of attempts to enter each maze arm. In FIG. 6, the spontaneous alternation indexes revealed in the Y-maze test correlate with the spatial cognition memory and short-term memory functions of the mouse, where the symbol * denotes a statistically significant difference (*P<0.05; N=9 in each group), and the notation N.S. denotes no statistically significant difference. The Y-maze test also reveals that thermal cycling-hyperthermia (TC-HT) surpasses continuous hyperthermia (HT) in mitigating the degeneration of cognition capability of the mouse with dementia in the Y-maze test.

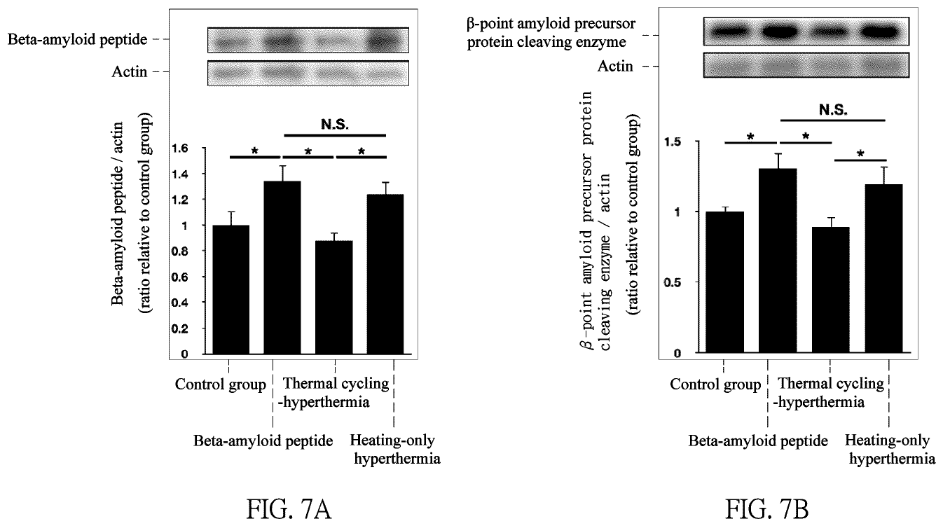

The completion of the Y-maze test is followed by sacrificing the mouse and taking out part of its brain tissue to undergo processing. Then, the quantity of specific proteins in the brain cells of the mouse is analyzed with Western blot technique as shown in FIG. 7A through FIG. 9B. First, the effect of thermal cycling-hyperthermia (TC-HT) and continuous hyperthermia (HT) on the expression of beta-amyloid peptide (Aβ) and β-site amyloid precursor protein cleaving enzyme 1 (BACE1) in the hippocampus of the mouse is analyzed as shown in FIG. 7A and FIG. 7B. Therefore, FIG. 7A shows the statistical result performed with Western blot analysis on Aα expression in the hippocampus of the mouse in the same four test groups as the test depicted by FIG. 6 and a representative Western blot band of Aβ, whereas FIG. 7B shows the statistical result performed with Western blot analysis on BACE1 in the hippocampus of the mouse in the same four test groups as the test depicted by FIG. 6 and a representative Western blot band of BACE1. The results reflect the production and accumulation of Aα in the hippocampus of the mouse in different test groups, where β-actin functions as a Western blot loading control protein. The results reveal that thermal cycling-hyperthermia (TC-HT) surpasses continuous hyperthermia (HT) in reducing the protein expression of Aα and BACE1 in the hippocampus of the mouse with dementia.

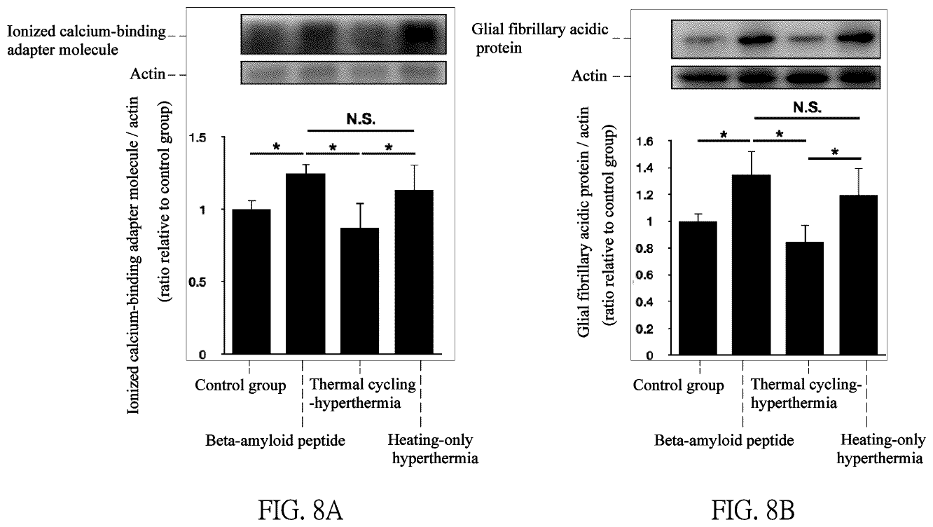

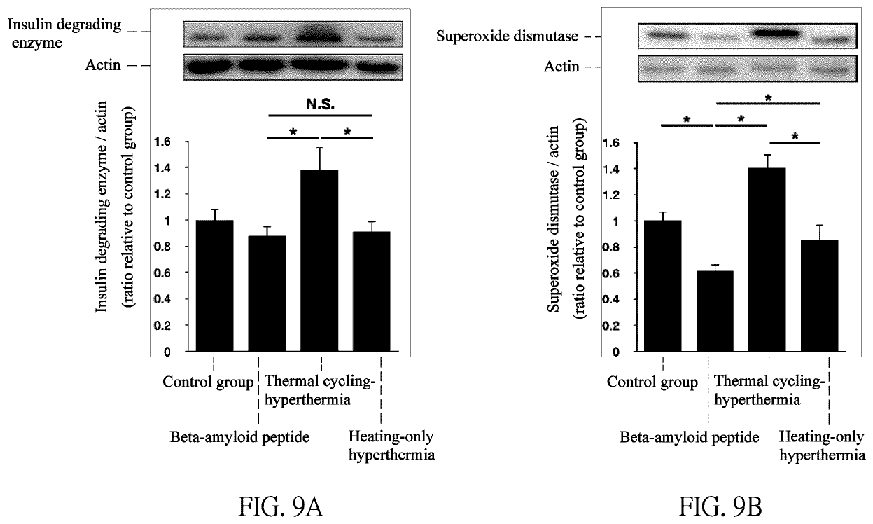

As shown in FIG. 8A and FIG. 8B, the disclosure involves exploring the effect of thermal cycling-hyperthermia (TC-HT) and continuous hyperthermia (HT) on the expression of ionized calcium-binding adapter molecule 1 (Iba-1) and glial fibrillary acidic protein (GFAP) indicative of neuroinflammation in the hippocampus of the mouse. Therefore, FIG. 8A shows the statistical result performed with Western blot analysis on Iba-1 in the hippocampus of the mouse in the same four test groups as the test depicted by FIG. 6 and a representative Western blot band of Iba-1, whereas FIG. 8B shows the statistical result performed with Western blot analysis on GFAP in the hippocampus of the mouse in the same four test groups as the test depicted by FIG. 6 and a representative Western blot band of GFAP. The results reflect the anti-neuroinflammation capability in the hippocampus of the mouse in different test groups. The results reveal that thermal cycling-hyperthermia (TC-HT) surpasses continuous hyperthermia (HT) in reducing the protein expression of Iba-1 and GFAP indicative of neuroinflammation in the hippocampus of the mouse with dementia. As shown in FIG. 9A and FIG. 9B, the disclosure involves exploring the effect of thermal cycling-hyperthermia (TC-HT) and continuous hyperthermia (HT) on the expression of insulin degrading enzyme (IDE) and superoxide dismutase (SOD2) for neurological protective proteins in the hippocampus of the mouse. Therefore, FIG. 9A shows the statistical result performed with Western blot analysis on IDE in the hippocampus of the mouse in the same four test groups as the test depicted by FIG. 6 and a representative Western blot band of IDE, whereas FIG. 9B shows the statistical result performed with Western blot analysis on SOD2 in the hippocampus of the mouse in the same four test groups as the test depicted by FIG. 6 and a representative Western blot band of SOD2. The results reflect the capability of degradation of Aα and the antioxidation capability in the hippocampus of the mouse in different test groups. The results reveal that thermal cycling-hyperthermia (TC-HT) surpasses continuous hyperthermia (HT) in increasing the expression of IDE and SOD2 for the neurological protective proteins in the hippocampus of the mouse with dementia.

The results shown in FIG. 6 through FIG. 9B indicate that the heating operation brought about with the thermal effect logic 12 executed by the control unit 11 is the thermal cycling-hyperthermia (TC-HT), and the resultant effect is the best. Thus, according to the disclosure, the heating device 10 for imposing intermittent thermal effect on neural tissue of an animal or a human body in vivo is effective in mitigating the effect of thermal stress and irritation on causing injuries to neural tissue continuously, reducing the likelihood of causing injuries to neural tissue, and allowing the thermal effect to last longer. Furthermore, the disclosure is effective in enabling activation and recovery of neural tissue when the thermal effect is imposed on neural tissue.

Although the first embodiment is exemplified by an animal, it is inferable that the device of the disclosure is also applicable to a human body.

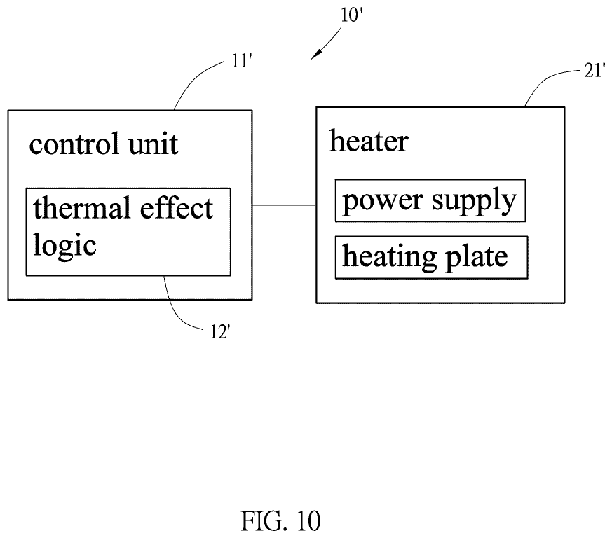

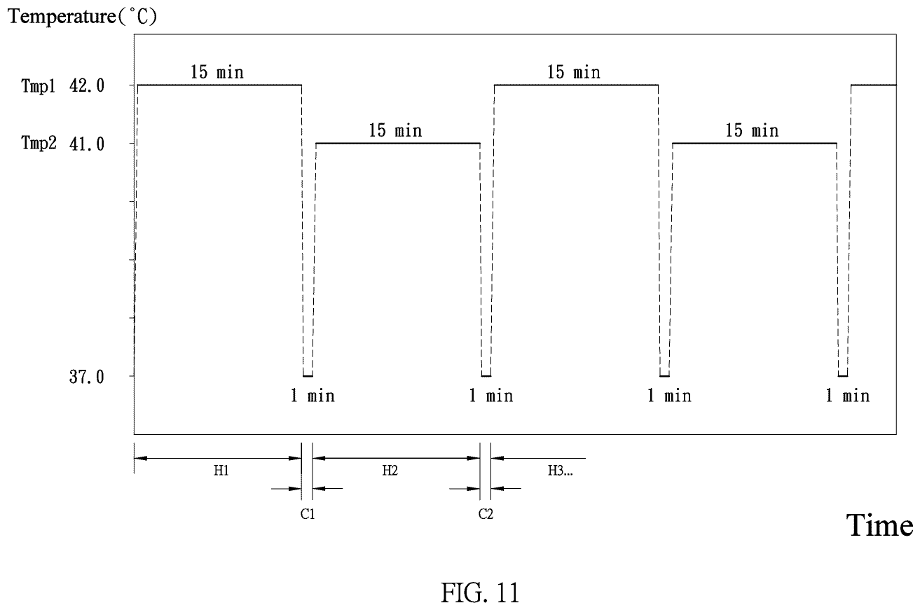

As shown in FIG. 10 and FIG. 11, the second preferred embodiment of the disclosure provides a heating device 10′ for imposing intermittent thermal effect on neural tissue of an animal or a human body in vivo, which is distinguished from the heating device 10 for imposing intermittent thermal effect on neural tissue of an animal or a human body in vivo in the first embodiment by technical features as follows:

The second embodiment lacks the temperature sensor of the first embodiment. In the absence of any temperature sensor, the temperature relationship between the heating power and the heated area 92 of a sample is obtained according to experimental data of the sample when a setting process is performed prior to delivery. Afterward, users only need to access the heating power in order to allow the temperature of the heated area 92 of an animal in vivo to reach the preset temperature. However, the temperature controlling effect of the second embodiment is less precise than the first embodiment; users may choose between the first embodiment and the second embodiment as needed.

In addition, regarding the thermal effect logic 12′, a first target temperature Tmp1, i.e., 42° C., is set in the first heating session H1, whereas a second target temperature Tmp2, i.e., 41° C., is set in the second heating session H2. Thus, the first target temperature Tmp1 is different from the second target temperature Tmp2. If the heating target temperature is relatively low (for example, the second target temperature Tmp2 is 41° C. and thus is lower than the first target temperature Tmp1), the effect and expression of heat shock proteins triggered by its heating up neural tissue will be relatively low, and the injuries it causes to the neural tissue will be relatively mild. Therefore, the target temperatures of the heating sessions are set to meet user needs or choices.

Owing to the first embodiment, it is inferable that the second embodiment is also effective in mitigating the effect of the thermal stress and irritation on causing injuries to neural tissue continuously, reducing the likelihood of causing injuries to neural tissue, and allowing the thermal effect to last longer than the aforesaid prior art. Furthermore, the other technical features and achievable advantages of the second embodiment are substantially the same as those of the first embodiment and thus are, for the sake of brevity, not reiterated.

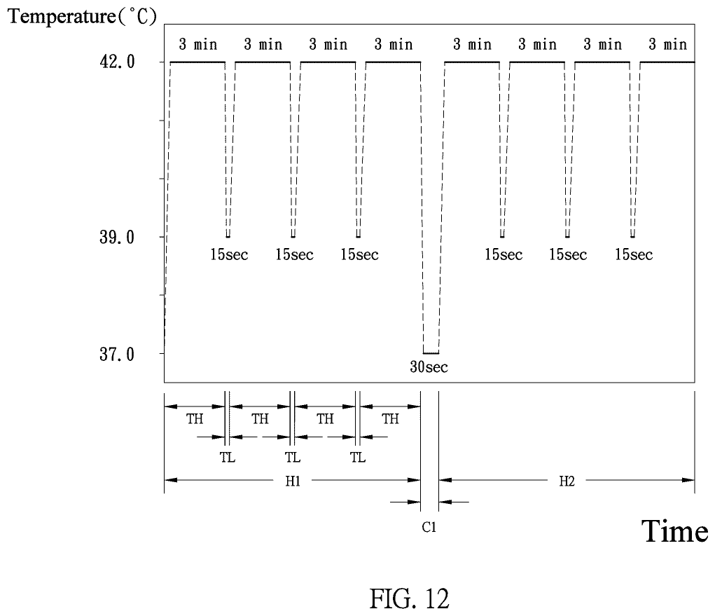

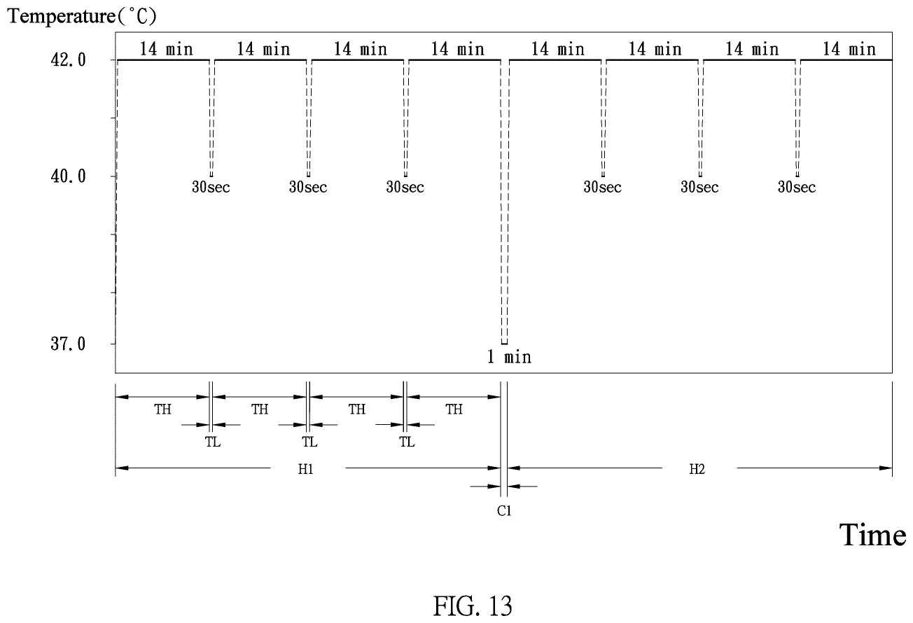

As shown in FIG. 12 and FIG. 13, the third preferred embodiment of the disclosure provides a heating device for imposing intermittent thermal effect on neural tissue of an animal or a human body in vivo, which is distinguished from the first embodiment by technical features as follows:

Regarding the thermal effect logic 12 (see FIG. 1), the power of the heater 21 is fine-tuned to sequentially form at least one high temperature session TH and at least one sub-high temperature session TL, preferably a plurality of high temperature sessions TH and a plurality of sub-high temperature sessions TL in practice, within the first heating session H1 and the second heating session H2 or any subsequent heating sessions. In each of the high temperature sessions TH, the heated area 92 (see FIG. 2) is heated up to reach a target temperature, for example, 42° C., and then stay above or at the target temperature. It is because if the target temperature is lower than 42° C. as is in the second embodiment, it will be possible to adjust the heating power of the heater 21 while the temperature is being maintained and thus slightly increase the temperature, causing the temperature of the heated area 92 to be higher than the target temperature. In each of the sub-high temperature sessions TL, the heated area 92 is not heated up or is heated up with a relatively low power such that the temperature of the heated area 92 decreases from the target temperature; then, after the temperature of the heated area 92 has decreased to a predetermined temperature no lower than 39° C., for example, 39° C., the heated area 92 is heated up with a low power to stay at the predetermined temperature, alternating in the order of high temperature, sub-high temperature, high temperature, and sub-high temperature. Referring to FIG. 12, each of the high temperature sessions TH lasts for 3 minutes, and each of the sub-high temperature sessions TL lasts for 15 seconds. Each high temperature session TH preferably lasts at least 3 minutes, and each sub-high temperature session TL lasts 5 seconds to 2 minutes. In addition, although FIG. 12 shows that all the high temperature sessions TH last for the same time period for exemplary purposes, they may last for different time periods as needed.

As shown in FIG. 13, in a variant embodiment, the target temperature in each high temperature session TH in the first heating session H1 is 42° C. and lasts for 14 minutes, whereas the predetermined temperature in each sub-high temperature session TL in the first heating session H1 is 40° C. and lasts for 30 seconds. Therefore, the first heating session H1 lasts almost 60 minutes as needed. Since the maximum temperature (42° C.) for the thermal effect in the disclosure is lower than the maximum temperature (46° C.) of the aforesaid prior art and approximates to high fever's temperature (41.5° C. or higher), temperature in the first cooling session C1 also further decreases to a human body's normal temperature, i.e., 37° C., allowing the neural tissue to take an appropriate low-temperature break. Therefore, it is reasonable to infer that the total duration of a heating session of the disclosure can exceed 30 minutes and thus approximate to 60 minutes without causing injuries to the neural tissue but still impose recovery effect on the neural tissue. The cooling session still lasts 5 seconds to 3 minutes.

Unlike the first and second embodiments, the aforesaid variant embodiment is characterized in that each heating session features a temperature fluctuation state of high temperature and sub-high temperature to allow the neural tissue to undergo temperature fluctuation in a high temperature state instead of staying at a target temperature so as to intermittently and appropriately reduce the temperature in the high temperature state, mitigate the effect of thermal stress and irritation on causing injuries to the neural tissue continuously, reduce the discomfort caused to patients receiving thermal therapy, and enable the thermal effect to last longer. The other technical features and achievable advantages of the third embodiment are substantially the same as those of the first embodiment and thus are, for the sake of brevity, not reiterated.

In conclusion, a heating device for imposing intermittent thermal effect on neural tissue of an animal or a human body in vivo according to the aforesaid three embodiments of the disclosure is operated to execute a thermal effect logic 12 and thereby impose thermal effect on neural tissue to mitigate the effect of thermal stress and irritation on causing injuries to the neural tissue continuously, reduce the likelihood of causing injuries to neural tissue, and allow the thermal effect to last longer than the aforesaid prior art.

What is claimed is:

1. A heating device for imposing intermittent thermal effect on neural tissue of an animal or a human body in vivo, comprising:
a control unit having a thermal effect logic to be executed by the control unit; and
a heater electrically connected to the control unit, the control unit executing the thermal effect logic to controllably cause the heater to heat up a portion of neural tissue of an animal or a human body in vivo, wherein an area to be heated is defined as a heated area,
wherein the thermal effect logic comprises:
sequentially, at least one first heating session is followed by a first cooling session, and the first cooling session is followed by a second heating session, the first or second heating session allowing the control unit to controllably cause the heater to perform a heating process, the first cooling session allowing the control unit to controllably cause the heater to not perform any heating process or to perform a heating process with low power, wherein the heating process performed with low power uses heating power lower than the heating power used in the first heating session,
the first heating session entails heating up the heated area to cause the heated area to stand above 39° C. and stay between 39° C. and 42° C., with the first heating session lasting for 3 to 60 minutes,
the first cooling session entails cooling down the heated area to cause the heated area to stand below 39° C. and stay between 37° C. and 39° C., with the first cooling session lasting for 5 seconds to 3 minutes, and
the second heating session entails heating up the heated area to cause the heated area to stand above 39° C. and stay between 39° C. and 42° C., with the second heating session lasting for 3 to 60 minutes,
wherein the first heating session and the second heating session each last longer than the first cooling session.

2. The heating device for imposing intermittent thermal effect on neural tissue of an animal or a human body in vivo according to claim 1, wherein, regarding the thermal effect logic, during the first heating session and the second heating session the heated area is heated up to stand at a target temperature and then stay at the target temperature until the first heating session and the second heating session end.

3. The heating device for imposing intermittent thermal effect on neural tissue of an animal or a human body in vivo according to claim 1, wherein, regarding the thermal effect logic, power of the heater is fine-tuned to sequentially form at least one high temperature session and at least one sub-high temperature session within the first heating session and the second heating session, in the high temperature session the heated area is heated up to reach a target temperature and then stay above or at the target temperature, and in the sub-high temperature session the heated area is not heated up or is heated up with a relatively low power such that the temperature of the heated area decreases from the target temperature; then, after the temperature of the heated area has decreased to a predetermined temperature no lower than 39° C., the heated area stays at the predetermined temperature, wherein high temperature, sub-high temperature, high temperature, and sub-high temperature sequentially alternate when both the at least one high temperature session and the at least one sub-high temperature session are in a plural number.

4. The heating device for imposing intermittent thermal effect on neural tissue of an animal or a human body in vivo according to claim 3, wherein the high temperature session lasts for a time period no shorter than 3 minutes, and the sub-high temperature session lasts 5 seconds to 2 minutes.

5. The heating device for imposing intermittent thermal effect on neural tissue of an animal or a human body in vivo according to claim 3, wherein two different high temperature sessions in the first heating session last for the same time period or different time periods.

6. The heating device for imposing intermittent thermal effect on neural tissue of an animal or a human body in vivo according to claim 1, wherein, regarding the thermal effect logic, the heated area is heated up to a first target temperature in the first heating session, and the heated area is heated up to a second target temperature in the second heating session, with the first target temperature being different from the second target temperature.

7. The heating device for imposing intermittent thermal effect on neural tissue of an animal or a human body in vivo according to claim 1, wherein, regarding the thermal effect logic, in the first heating session the heated area is heated up from a human body temperature to a target temperature within 2 minutes, with the target temperature being not higher than 42° C.

8. The heating device for imposing intermittent thermal effect on neural tissue of an animal or a human body in vivo according to claim 7, wherein, regarding the thermal effect logic, in the first heating session, heating up the heated area to the target temperature involves causing the temperature of the heated area to be higher than the target temperature by no greater than 0.5° C. and then reducing a heating power of the heater to cause the heated area to stay at the target temperature.

9. The heating device for imposing intermittent thermal effect on neural tissue of an animal or a human body in vivo according to claim 1, wherein, regarding the thermal effect logic, sequentially the second heating session is followed by a second cooling session and a third heating session.

10. The heating device for imposing intermittent thermal effect on neural tissue of an animal or a human body in vivo according to claim 1, further comprising a temperature sensor electrically connected to the control unit and adopted to sense a temperature of the heated area.

11. The heating device for imposing intermittent thermal effect on neural tissue of an animal or a human body in vivo according to claim 1, wherein the heater provides heat pulse energy with frequency of 5˜500 Hz, preferably 25˜50 Hz.

12. The heating device for imposing intermittent thermal effect on neural tissue of an animal or a human body in vivo according to claim 1, wherein the heater provides heat pulse energy with frequency of 40 Hz.

13. The heating device for imposing intermittent thermal effect on neural tissue of an animal or a human body in vivo according to claim 1, wherein the heater is a focused ultrasound device for generating ultrasound to provide heat pulse energy, and enable the heat pulse energy to penetrate a skin of an animal or a human body in vivo through focusing technology, allowing the heat pulse energy to directly act on interior neural tissue of the animal or the human body.
